(19) United States
(12) Patent Application Publication
(10) Pub. No.: US 2023/0310717 A1
(43) Pub. Date: Oct. 5, 2023

(54) TISSUE PLASMINOGEN ACTIVATOR FORMULATION

(71) Applicant: CHIESI FARMACEUTICI S.p.A., Parma (IT)

(72) Inventor: Ray Anthony Mina ANOVER, Parma (IT)

(21) Appl. No.: 18/041,322

(22) PCT Filed: Aug. 6, 2021
(86) PCT No.: PCT/EP2021/071977

§ 371 Date: Feb. 10, 2023

Related U.S. Application Data
(60) Provisional application No. 63/063,770, filed on Aug. 10, 2020.

(51) Int. Cl. A61L 29/16; A61K 38/49; A61K 31/196

(52) U.S. Cl. CPC A61L 29/16; A61K 38/49; A61K 31/196; A61L 2300/254

(57) ABSTRACT

The present disclosure relates to stable formulations of tissue-type plasminogen activator (tPA). In particular, the disclosure relates to specific new formulations, methods of preparing them and methods of using them.

### FIELD OF THE INVENTION

The present disclosure relates to stable formulations of tissue-type plasminogen activator (tPA). In particular, the disclosure relates to specific new formulations, methods of preparing them and methods of using them.

### BACKGROUND OF THE INVENTION

Plasminogen activators (PA) are endogenous serine proteases involved in a cascade of events leading to the dissolution of a blood clot. These proteins are classified in two distinct groups: urokinase-type plasminogen activator (uPA) and tissue-type plasminogen activator (tPA) (Nguyen et al., 1993, in Stability and Characterization of Protein and Peptide Drugs: Case Histories, Plenum Press, New York). Tissue plasminogen activator (tPA), in particular, is a protein involved in the breakdown of blood clots. It is a serine protease (EC 3.4.21.68) found on endothelial cells, the cells that line the blood vessels. As an enzyme, it catalyzes the conversion of plasminogen to plasmin, the major enzyme responsible for clot breakdown. Human tPA has a molecular weight of ˜70 kDa in the single-chain form.

Recombinant tPA include alteplase (Nguyen et al., 1993, in Stability and Characterization of Protein and Peptide Drugs: Case Histories, Plenum Press, New York), reteplase: a recombinant mutant of alteplase associated with a longer in vivo half-life than alteplase (N. Engl. J. Med, 1997, vol. 337, p. 1118-1123), tenecteplase (Dillon et al., ibid), and desmoteplase (Li et al., 2017, Medicine (Baltimore), vol. 96, e6667).

Recombinant tPAs have been developed as medicines suitable for thrombolysis. In particular, recombinant tPAs have been developed for the treatment or prevention of certain diseases that feature blood clots, such as pulmonary embolism, myocardial infarction, and stroke. A common use is for ischemic stroke. Recombinant tPAs can either be administered systemically, e.g. in the case of acute myocardial infarction or acute ischemic stroke, or administered through an arterial catheter directly to the site of occlusion in the case of certain thrombi. The dosages to be administered normally vary greatly between systemic administration and local administration, such as through a catheter.

Recombinant tPAs, in general, like many other biological active ingredients, are associated with poor long-term stability at room temperature. Indeed, many biological active ingredients, in general, are prone to undesired instability, including degradation, when stored in liquids, such as aqueous solutions. This poses a challenge not only to providing ready-to-use formulations of such biological active ingredients but can also be associated with disadvantages during the production and storage of such biologicals. Recombinant tPAs have not been made available as stable liquids and or otherwise in room temperature-stable form; they are usually offered in lyophilized form (lyophilizate), which typically need to be stored at refrigerated temperatures. Lyophilized Cathflo, for example, should be stored at refrigerated temperatures (2° C.-8° C./36° F.-46° F.). Cathflo should be reconstituted immediately before use.

It would be desirable to have a stable composition of recombinant tPA that can be stored and commercialized at room temperature. It would also be desirable that such specific stable composition of recombinant tPA can be conveniently manufactured and formulated without undergoing undesired degradation during the manufacturing process. It would also be desirable to obtain a tPA formulation that can be readily used, e.g. after reconstitution, in catheters, such as venous access devices.

### SUMMARY OF THE INVENTION

The present disclosure relates to novel formulations of tissue plasminogen activator (tPA) which are stable to storage and/or stress, and which are suitable for treating a patient with occluded catheters. In particular, the amount of the tPA in the formulations according to the present disclosure is such that the liquid formulation as described herein can be directly instilled into a patient's occluded catheter, thereby aiming at clearing the catheter occlusion.

In a first aspect, the present disclosure relates to a liquid composition comprising:
i. about 0.15 to 0.25 units (U)/mL tissue plasminogen activator (tPa),
ii. about 1 mM to 10 mM tranexamic acid,
iii. about 2.5 to 5.0% (w/w) sucrose; (preferably about 3.0% (w/w),
   wherein the formulation has a pH of 6.0 to 8.0, preferably 7.0 to 8.0, preferably 7.5.

Optionally, also a nonionic surfactant and a pH buffering agent are comprised in said composition. Such composition is defined in comprising the following:
i. about 0.15 to 0.25 units (U)/mL tissue plasminogen activator (tPa),
ii. about 1 mM to 10 mM tranexamic acid,
iii. about 2.5 to 5.0% (w/w) sucrose; (preferably about 3.0% (w/w),
iv. optionally a nonionic surfactant,
v. optionally a pH buffering agent;
   wherein the formulation has a pH of 6.0 to 8.0, preferably 7.0 to 8.0, preferably 7.5.

In a second aspect, the disclosure relates to an essentially water-free composition obtainable by removing water from the composition of the first aspect, preferably by lyophilization.

In a third aspect, the present invention relates to a method of treating a patient suffering from impaired functionality of a catheter, wherein the method is characterized in that the liquid formulation of the first aspect of the present disclosure is instilled into an occluded catheter of the patient. Preferably, the catheter is a central venous access device (CVAD), and the composition is directly instilled into the CVAD.

In a fourth aspect, the present disclosure also relates to a kit comprising the essentially water free composition of the first aspect as well as water for reconstitution.

In a fifth aspect, the present disclosure also relates to a method of obtaining the composition of the first aspect, wherein the method essentially consists of admixing water to the composition of the second aspect.

In a sixth aspect, the present disclosure also relates to the use of the composition of the first and second aspects to restore the functionality of a catheter.

### DETAILED DISCLOSURE

This specification in its entirety, together with the claims and the figures, discloses specific and/or preferred embodiments and variants of the individual features of the invention. The present invention also contemplates as particularly preferred embodiments those embodiments, which are generated by combining two or more of the specific and/or preferred embodiments and variants described herein for the present invention. Thus, the present disclosure also includes all of the entities, compounds, features, steps, methods, details of administration or compositions referred to or indicated in this specification, individually or collectively, and any and all combinations or any two or more of said entities, compounds, features, steps, methods, details of administration or compositions. Thus, unless specifically stated otherwise herein or the context requires otherwise, reference to a single entity, compound, feature, step, method, detail of administration or composition shall be taken to encompass one and a plurality (i.e. more than one, such as two or more, three or more or all) of those entities, compounds, features, steps, methods, details of administration or compositions. Unless specifically stated otherwise or the context requires otherwise, each embodiment, aspect and example disclosed herein shall be taken to be applicable to, and combinable with, any other embodiment, aspect or example disclosed herein.

The person of ordinary skill in the art will appreciate that the invention described herein is susceptible to variations and modifications other than those specifically described. Thus, the present disclosure is not limited in scope by the specific embodiments described herein, which are provided herein for the purposes of illustration and of exemplification. Functionally or otherwise equivalent entities, compounds, features, steps, methods, details of administration or compositions are within the scope of the present disclosure. It will be apparent to the person of ordinary skill in the art that the present disclosure includes all variations and modifications of the entities, compounds, features, steps, methods, details of administration or compositions literally described herein.

Each of the references cited herein (including all patents, patent applications, scientific publications, manufacturer's specifications, instructions, presentations, etc.), whether above or below, are hereby incorporated by reference in their entirety. Nothing herein is to be construed as an admission that the present invention would not be entitled to antedate a specific teaching and/or as an admission that a specific reference, other than the common general knowledge, would contain information sufficiently clear and complete for it to be carried out by a person skilled in the art without undue burden.

Generally, unless specifically defined otherwise, all technical and scientific terms used herein have the same meaning as commonly understood by one of ordinary skill in the art (e.g., in genetics, molecular biology, gene expression, cell biology, cell culture, immunology, medicine, chromatography, protein chemistry, and biochemistry). Textbooks and review articles published e.g. in English typically define the meaning as commonly understood by one of ordinary skill in the art.

The expression “and/or”, e.g., “X and/or Y” shall be understood to mean either “X and Y” or “X or Y” and shall be taken to provide explicit disclosure of “and”, of “or” and of both meanings (“and” or “or”).

As used herein, unless specified otherwise, the terms “about”, “ca.” and “substantially” all mean approximately or nearly, and in the context of a numerical value or range set forth herein preferably designates +/−10%, more preferably +/−5%, more preferably +/−2%, more preferably +/−1%, around the numerical value or range recited or claimed. “Substantially”, used in relation to the absence of a particular feature, step, time interval, or the like, normally means that the invention is carried out in such a manner that the respective feature, step, time interval, or the like is about absent, or kept to the necessary handling minimum.

Unless expressly specified otherwise, the word “comprise”, or variations such as “comprises” or “comprising” is used in the context of the present document to indicate that further members may optionally be present in addition to the members of the list introduced by “comprising”. It is, however, contemplated as a specific embodiment of the present invention that the term “comprising” encompasses the possibility of no further members being present, i.e. for the purpose of this embodiment “comprising” is to be understood as having the meaning of “consisting of”.

Unless expressly specified otherwise, all indications of relative amounts regarding the present invention are made on a weight/weight basis. Indications of relative amounts of a component characterized by a generic term are meant to refer to the total amount of all specific variants or members covered by said generic term. If a certain component defined by a generic term is specified to be present in a certain relative amount, and if this component is further characterized to be a specific variant or member covered by the generic term, it is meant that no other variants or members covered by the generic term are additionally present such that the total relative amount of components covered by the generic term exceeds the specified relative amount; more preferably no other variants or members covered by the generic term are present at all.

Tissue plasminogen activator (tPA), as used herein, refers to a serine protease capable to catalyze the conversion of plasminogen to plasmin. tPA enzyme is suitable to catalyze the breakdown of fibrin clots (fibrinolysis). Without wishing to be bound to a particular theory, it is envisaged that the tPA acts by targeting fibrin (which causes blood to clot), dissolving the thrombus (blood clot). In humans, for example, tPA is found on endothelial cells (cells that line the blood vessels), but the term is not limited to human origin. Human and non-human tPAs are comprised in the term tPA as used herein. The tPA may be recombinant or not.

Recombinant tPA, as used herein, refers to any tissue plasminogen activator that has been recombinantly expressed or is available from a recombinant source. Recombinant tPA include, without limitation, alteplase (Dillon et al., 2019, Adv. Emerg. Nurs. J., vol. 41, p. 271-278), reteplase (Dillon et al., 2019, ibid), tenecteplase (Dillon et al., ibid), duteplase (Kalbfleisch et al., 1992, Am. J. Cardiol., vol. 69, p. 1120-1127), and desmoteplase (Li et al., 2017, Medicine (Baltimore), vol. 96, e6667).

An active pharmaceutical ingredient, as used herein, is a substance suitable to be used in the manufacture of a drug (medicinal) product and that, when used in the production of a drug, becomes an active ingredient of the drug product.

Herein, the term active ingredient is used to refer to any component of a drug product suitable to furnish pharmacological activity or other direct effect in the diagnosis, cure, mitigation, treatment, or prevention of disease, or to affect the structure or any function of the body of humans or other animals. Active ingredients include those components of the product that may undergo chemical change during the manufacture of the drug product and be present in the drug product in a modified form intended to furnish the specified activity or effect.

Within the context of the present invention, preferred active ingredient is selected from thrombolytic agents derived from human tissue plasminogen activator. The most preferred active ingredient is reteplase (Wooster et al., 1999, Ann. Pharmacother., vol 33, p. 318-324).

Drug product, as used herein, generally refers to a finished dosage form, for example, a tablet, capsule, a powder or solution that contains an active pharmaceutical ingredient, generally, but not necessarily, in association with inactive ingredients. In a specific context, drug product refers to the composition according to the first aspect and/or to the composition according to the second aspect of the present disclosure.

The term drug substance is used interchangeably with active ingredient.

According to the present invention, specific compositions comprising tissue plasminogen activator (tPA) are provided. Thus, specifically in the context of the present invention, tPA can also be referred to as drug substance.

When referring to compositions of tPA, the terms “formulation” and “composition” can be used interchangeably and define compositions in any state of aggregate that comprise tPA plus one or more excipients, such as those generally described herein.

More particularly, the present disclosure relates to a formulation of tissue plasminogen activator (tPA). Several embodiments of such compositions are provided herein. In its essentially water-free form, e.g. freeze-dried, the formulation is stable at room temperature. The essentially water-free formulation is obtainable e.g. by freeze-drying of a liquid formulation. Said liquid formulation is stable to stress conditions frequently encountered in commercial production of biopharmaceuticals, such as freeze-thaw stress in particular. Thus, in consideration of all these advantages in combination, the pair of the essentially water-free formulation, which is storage stable, and the liquid formulation, which is resistant to handling stress during production, together provide a suitable pharmaceutical formulation which can be readily prepared. The disclosure also contemplates a method of treating a patient suffering from impaired functionality of a central venous access device (CVAD), comprising administering to the patient the liquid composition of the present disclosure. For such purpose, the essentially water-free formulation of the invention may be reconstituted with water.

In general, whether or not specifically mentioned, it is generally preferred throughout all aspects and embodiments of the present disclosure that the water is water at a high purity grade, more preferably water for injection (WFI). In particular, sterile water for injection (sWFI) is preferred.

In the present disclosure, the active pharmaceutical ingredient tPA (e.g. reteplase) is formulated in a composition comprising sucrose and tranexamic acid as described herein. More particularly, the tPA is herein preferably formulated in an excipient system comprising Tranexamic acid (TA), phosphate and sucrose (together: TAPS) components). Although TAPS containing reteplase formulations have been previously known (see Comparative Example 3), the formulation of the present invention is not only particularly stable both in liquid and in essentially water-free form, at a ratio of components entirely different from that known previously (Comparative Example 3), but is also directly suitable for instilment into catheters, not requiring dilution. The compositions of the present disclosure are markedly distinguished from known tPA compositions, in particular in the ratio of tPA to other constituents of the composition.

### Liquid Composition

In a first aspect, the present disclosure relates to a liquid composition comprising:
i. about 0.15 to 0.25 units (U)/mL tissue plasminogen activator (tPA),
ii. about 1 mM to 10 mM tranexamic acid,
iii. about 2.5 to 5.0% (w/w) sucrose; (preferably about 3.0% (w/w),
   wherein the formulation has a pH of 6.0 to 8.0, preferably 7.0 to 8.0, preferably 7.5.

Optionally, also a nonionic surfactant and a pH buffering agent are comprised in said composition. Such composition is defined in comprising the following:
i. about 0.15 to 0.25 units (U)/mL tissue plasminogen activator (tPa),
ii. about 1 mM to 10 mM tranexamic acid,
iii. about 2.5 to 5.0% (w/w) sucrose; (preferably about 3.0% (w/w),
iv. optionally a nonionic surfactant,
v. optionally a pH buffering agent;
   wherein the formulation has a pH of 6.0 to 8.0, preferably 7.0 to 8.0, preferably 7.5.

The individual constituents and their relationship and relevance will now shortly be described.

The liquid composition typically comprises water as a liquid. Preferably, the liquid formulation is a water-based liquid formulation.

In one embodiment, said composition is liquid. In another embodiment, said composition is frozen. A preferred temperature range of the frozen composition is in the range of −10 to −80 degrees Celsius, such as about −20 degrees Celsius.

### Recombinant Tissue Plasminogen Activator

Recombinant tissue plasminogen activator (rtPA), is a recombinant form of tissue plasminogen activator (tPA), and can also be referred to as rPA.

Herein, the rtPA can also be referred to as API (active pharmaceutical ingredient). The tPA suitable for use in the composition according to the present disclosure is not particularly limited and include all known tPa, recombinant or otherwise, including alteplase (Nguyen et al., 1993, in Stability and Characterization of Protein and Peptide Drugs: Case Histories, Plenum Press, New York), reteplase: a recombinant mutant of alteplase associated with a longer in vivo half-life than alteplase (N. Engl. J. Med, 1997, vol. 337, p. 1118-1123), tenecteplase (Dillon et al., ibid), tenecteplase and desmoteplase (Li et al., 2017, Medicine (Baltimore), vol. 96, e6667).

Commercial formulations of rtPAs are available, but these do not fall under the present invention (Comparative Examples 1, 2 and 3).

Preferred tPAs in the present disclosure include alteplase and reteplase, whereby reteplase is particularly preferred. Reteplase is a particular rtPA, and is the most preferred rtPA herein. The structure, function, and production of reteplase have been described extensively in the literature, see e.g. Muhammadi et al., 2019, Adv. Biomed. Res. Vol. 8, 19. In some embodiments, the formulations of the present invention are obtainable from reteplase as exemplified in Comparative Example 3 (Retavase) or from reteplase drug substance.

Preferably, the tissue plasminogen activator (tPA) is reteplase. More preferably, according to the first aspect, the reteplase is contained at an amount of about 0.2 units (U)/mL. Expressed differently, it is preferred that the tPa, preferably reteplase, is present at about 0.3 mg/mL to about 0.4 mg/mL, and more preferably about 0.35 mg/mL.

Without wishing to be bound to a particular theory, it is envisaged that the tPA acts by targeting fibrin (which causes blood to clot), dissolving the thrombus (blood clot). When administered as described herein, the formulations described herein are useful for restoring function of the central venous access device (CVAD) in a patient. In general, tPA, such as e.g. alteplase and reteplase, is highly fibrin specific. Thus, preferably the tPA comprised in the compositions according to the present invention acts specifically on fibrin-rich clots in catheter occlusion.

### Tranexamic Acid

As experimentally evidenced herein, tranexamic acid has a beneficial effect on the thermodynamic stability of reteplase (see Example 1). The thermodynamic stability is enhanced by increasing the tranexamic acid concentration. In particular, higher concentrations of tranexamic acid can be beneficial (see Example 1). However, at concentrations as low as 1 mM stability is already observed. Thus, a particularly preferred amount of tranexamic acid in the liquid formulation according to the first aspect is about 1 mM.

### Sucrose

As shown in the present disclosure, higher sucrose levels seemed to have a slightly beneficial effect (Example 1), however, very high sucrose level (8%) might complicate the lyophilization process without providing any added beneficial effect (Example 2).

For efficient lyophilization, the 3% sucrose formulation variant is the most appropriate candidate to move forward (see Example 4).

The relative amount of sucrose, with respect to tPA, according to the present disclosure is markedly different to the relative amount of sucrose, with respect to tPA, as known in the art (see e.g. Example 6). Without wishing to be bound to any particular theory, it is believed that this is crucial providing the stability advantages of the formulations according to the present disclosure.

### Nonionic Surfactant

Preferably, the liquid composition according to the first aspect comprises a nonionic surfactant. Preferably, the nonionic surfactant is polysorbate 80 (PS80). A preferred concentration of the PS80 in the liquid formulation is about 0.001 (w/w) and 0.1% (w/w), preferably about 0.01% (w/w), still preferably about 0.02% (w/w), preferably about 0.03% (w/w), preferably about 0.04% (w/w), preferably about 0.045% (w/w), preferably about 0.06% (w/w), preferably about 0.0675% (w/w), preferably about 0.07% (w/w), preferably about 0.08% (w/w), preferably about 0.09% (w/w).

### pH

The pH of the liquid composition according to the first aspect is preferably in the range of 6.0 to 8.0, preferably 7.0 to 8.0, still preferably 7.5. As shown experimentally, variation of pH towards higher values (pH 7.5) showed a clear beneficial effect with regard to colloidal stability (see Example 1).

To that end, preferably, the liquid composition comprises also a pH buffering agent. More preferably, said pH buffering agent is a potassium phosphate buffering system. Preferred concentrations of potassium include the range of 20-30 mM potassium, more preferably about 26 mM potassium, with the appropriate amount of potassium counterion to guarantee a pH within the range above. For example, concentrations of potassium phosphate include the range of 20-30 mM potassium phosphate, more preferably about 26 mM potassium phosphate.

It is a preferred advantage of the liquid composition of the first aspect that said composition is stable e.g. at refrigerated temperatures for at least 3 months, preferably at least 6 months.

The liquid formulation is also stable to freeze-thaw stress. Therefore, if necessary, the liquid formulation may be frozen and thawed multiple times, if necessary. This is very useful during production, and allows frozen storage of both bulk product as well as aliquoted samples, such as in the vials contemplated herein, wherein thawing and re-freezing is possible without compromising quality.

### Preparation of the Liquid Formulations

Even though there may be batch-to-batch variety in bioactivity of the tPA substance to be used in formulation of the compositions according to the present invention, the particular amount of tPA is adjusted in the manufacturing process, so that the Units filled in each vial are always within the limits of the present disclosure. In other words, even if there will be batch to batch variability of clot lysis activity, this is not a concern since it will be adjusted/formulated accordingly in the subsequent manufacturing steps prior to filling of vials (aliquots) and lyophilization.

### Aliquots

In some embodiments, the liquid composition is comprised in a single use vial, also termed “aliquot”. A single use vial filled with the composition according to the present disclosure can also be referred to as unit dosage composition, or dosage unit.

In particular, the amount of the tPA in the formulations according to the present disclosure is such that the liquid formulation as described herein can be directly instilled into a patient's occluded catheter, thereby aiming at clearing the catheter occlusion.

Preferably, the formulation of the present invention is aliquoted such that it comprises 0.4 Units of clot lysis activity in each vial of formulation according to the present invention.

Such single use vial comprising the liquid formulation is obtainable by filling a vial with the liquid formulation, or alternatively, by admixing water to the essentially water-free formulation according to the second aspect of the invention, which will now be described.

### Certain Advantages of the Liquid Formulation

Thus, in a first aspect, the present invention relates to a liquid composition as herein described.

Chemical degradation the liquid presentation of the drug product (DP) was not observed for samples exposed to freeze-thaw stress (see also e.g. Example 2), accounting for suitability for a freeze-dried presentation of the drug product. In other words, the liquid formulation is stable to freeze-thaw stress, which represents a major advantage during production, where repeated freeze-thaw cycles are often a step in the manufacturing process.

The amidolytic activity of the tPA remains essentially unaffected when exposed to freeze-thawing (see e.g. Example 2).

To put certain advantages of the liquid formulation into context, the following points, among others, are noteworthy.

First, in general, degradation for protein molecules is slower at lower temperatures; therefore, storage at lower temperature is in many cases a preferred option for extending shelf life. In particular, it is recognized that the storage at subzero temperatures mitigates potential risks associated with liquid storage, such as degradation and shipping stress, making it a suitable option for long-term storage (Rayfield et al., 2017, J. Pharm Sci., vol. 106, p. 1944-1951).

However, slower rates of freezing and thawing, particularly in large storage containers (>2 L) can lead to greater cryo-concentration (i.e. exclusion of solute molecules), which results in zones of higher protein and excipient concentrations and changes to the desired formulation, pH, and excipient concentration. These conditions can negatively impact product quality. Thus, freezing and thawing can normally affect the target product profile.

While freezing and thawing is typically used for storage of drug substance, it may also be important for drug product (finished pharmaceutical formulation), such as e.g. for clinical drug product, to supply potentially lengthy clinical trials (Ho et al., 2008, Am. Pharmaceut. Rev., p. 1-6).

Thus, the fact that the composition according to the first aspect is stable to freeze-thaw cycles is associated with several advantages, which would not have been predictable by a person of ordinary skill in the art.

Further, as also described in more detail herein below, from the liquid composition of the present disclosure also the essentially water-free composition of the present disclosure can be readily obtained, e.g. by lyophilization as described in Example 4. In return, the liquid composition of the present disclosure can be directly obtained from the essentially water-free composition of the present disclosure, e.g. by addition of a liquid, preferably water, more preferably water for injection. Most preferably, the liquid to be added is sterile water for injection.

### Liquid Formulations

In order to achieve 0.4 Units of clot lysis activity in each vial of the formulations according to the present invention, it will be necessary to dilute the reteplase (e.g. Comparative Example 3) with the amount of each excipient based on the initial clot lysis activity of that particular batch. For illustration, if the reteplase active pharmaceutical ingredient (API) can be a range of 0.679 -0.721 mg, then the following estimated ranges may be suitable for the excipients.

Reteplase: 0.7 mg (possible range upon establishment of commercial manufacture: 0.679-0.721 mg so that's ±3%). Using the same ±3%, then the excipients range will potentially be,
Tranexamic acid: 1 mM (target 1 mM=0.31 mg, range: 0.30-0.32 mg)
Sucrose: 3% (target 3%=61 mg, range: 59-63 mg)
Polysorbate 80: 0.001-0.09% (for instance 1.9 mg, range: 1.84-1.96 mg)
Dipotassium hydrogen phosphate: target 8.36 mg, range: 8.11-8.61 mg
Phosphoric acid: target 0.69 mg, range: 0.67-0.71 mg

In one embodiment, said composition is liquid. In another embodiment, said composition is frozen. A preferred temperature of the frozen composition is about — 20 degrees Celsius.

### Essentially Water-Free Composition

In a second aspect, the disclosure relates to an essentially water-free composition obtainable by removing water from the composition of the first aspect, preferably by lyophilization.

Thus, except for the difference in the content of a liquid, preferably water, the essentially water-free composition of the second aspect corresponds to the liquid composition of the first aspect. Therefore, and for the sake of conciseness, the characteristics of the individual constituents of such compositions, such as sucrose, tranexamic acid, nonionic surfactant, pH, pH buffering agent, in particular, are not again described herein in the context of the second aspect. The respective disclosure of the first aspect should be read into the second aspect as well, as if it were explicitly recited here with respect to the second aspect as well. Thus, all above described embodiments of the first aspect are explicitly convertible into embodiments of the second aspect, whether explicitly so described herein or not. Merely for illustration, in the second aspect it is preferred that the tissue plasminogen activator (tPA) in the essentially water-free composition is reteplase. Such composition according to the second aspect is obtainable e.g. by freeze-drying a reteplase composition according to the first aspect.

It is a preferred advantage of the essentially water-free composition of the second aspect, said composition is stable at room temperature for at least 3 months, preferably at least 6 months. Even more preferably, the composition is stable at room temperature for at least 1 year, preferably at least two years, more preferably at least 3 years. Room temperature refers to the temperature range of about 20 to about 25 degrees Celsius, such as selected from the following: about 20 degrees Celsius, about 21 degrees Celsius, about 22 degrees Celsius, about 23 degrees Celsius, about 24 degrees Celsius, about 25 degrees Celsius.

In general, stability at room temperature can be predicted based on experimentally evidenced stability at harsher conditions, see Examples herein.

The essentially water-free composition of the second aspect is obtainable by removing water from the liquid formulation of the first aspect, such as described e.g. in Example 4. A suitable method for removing water is freeze-drying, also referred to as lyophilization. The composition will normally be obtained as a lyophilized substance (also referred to as lyophilization cake) in the vial where it was lyophilized. Preferably, the lyophilized substance is in the form of a powder.

Preferably, the essentially water-free composition of the second aspect is present in a single use vial. Preferably the single use vial has a volume capacity of 2 to 15 mL. This allows that, after reconstitution, said single use vial comprises approximately 2 ml of the liquid composition of the first aspect. Said volume is suitable for instillation, partially or completely, into a catheter of a human patient, as described herein. Thus, the liquid formulation obtained by reconstitution will not require dilution. This represents a major advantage over the theoretical use of commercial Retavase (Comparative Example 3) for such medical use, since significant dilution of the commercial Retavase would first be required. For the avoidance of doubt, reconstitution refers to admixing water (preferably water for injection) to the composition of the second aspect, thereby obtaining the composition of the first aspect.

Drug product is produced into a lyophilized form to give it the stable properties in room temperature. Thus, the essentially water-free composition according to the present disclosure can also be referred to as drug product. The drug product will normally need to be reconstituted with water, preferably water for injection (WFI), to be suitable in the method of treating a patient or a method to restore the functionality of a catheter, as described herein below.

Thus, in a second aspect, the present invention relates to an essentially water-free composition as herein described. Essentially water free means that the water content (w/w) is 2% or less, preferably 1% or less (for experimental support, see e.g. Example 5).

The freeze-dried composition of the second aspect and the essentially water-free composition of the second aspect are directly related to each other and are mutually convertible by addition or removal of water:
1. In one embodiment, the essentially water-free composition according to the second aspect is directly obtainable by (aliquoting, if necessary, and) freeze-drying the liquid composition according to the first aspect of the present invention.
2. In one embodiment, the liquid composition according to the first aspect is directly obtainable by admixing water and reconstituting the essentially water-free composition according to the second aspect of the present invention.

Thus, not only the e.g. freeze-dried composition according to the second aspect is associated with technical advantages, nor is only the liquid composition according to the first aspect associated with advantages: on top of all those advantages of the individual compositions, a further advantage resides in the direct convertibility of said composition. Thus, in the present invention, advantages in the process of manufacture of the respective compositions are linked to advantages in the product resulting from such process.

In one embodiment the essentially water-free composition is obtainable by freeze drying. Such formulation can be referred to as freeze-dried formulation.

As illustrated e.g. in Example 2, an essentially water-free (e.g. freeze-dried) presentation of formulations according to the present disclosure is stable for storage.

As described herein, the essentially water-free composition of the present disclosure can be readily obtained from the liquid composition of the present disclosure, e.g. by lyophilization as described in Example 4. The essentially water-free composition is stable (see Example 5).

The above compositions including all the compounds or at least one of the here above listed, can be generally prepared according to the procedure outlined in detail herein using generally known methods.

The essentially water-free composition according to the present invention is associated with several advantages over compositions known in the art. For example, Cathflo is a known formulation of a tPA for in-catheter use. Briefly, Cathflo contains the active ingredient alteplase, and excipients arginine, polysorbate 80, and phosphoric acid for adjusting pH. Although Cathflo™ is marketed as a lyophilized product, the lyophilisate must be stored at refrigerated temperature (2-8° C./36-46° F.), see Comparative Example 2.

In contrast, the essentially water-free composition according to the present invention is stable at room temperature. Further, as described herein, the essentially water-free composition according to the present invention is also directly obtainable by (aliquoting, if necessary, and) freeze-drying the liquid composition according to the present invention.

In one embodiment, the essentially water-free formulation is provided in a single-use lyophilized vial format. Specifically, it may be provided for reconstitution with water, such as in particular sterile water for injection (sWFI) suitable for and intended for catheter clearance.

In general, although there is a generic tendency that the stability of protein molecules can be extended by freezing to subzero temperatures, there is no general tendency as to the effects of freeze-thawing on the stability of protein molecules: even structurally relatively closely related protein molecules can differ significantly in their stability to freeze thaw exposure (Rayfield et al., 2017, J. Pharm Sci., vol. 106, p. 1944-1951). Therefore, the specific stability of the liquid formulation according to the present disclosure to freeze thaw exposure was not predictable based on the knowledge of the person of ordinary skill in the art. It was also not predictable that such lyophilized formulation would be stable at room temperature.

### Method of Treating a Patient

In a third aspect, the present invention relates to a method of treating a patient suffering from impaired functionality of a catheter, wherein the method is characterized in that the liquid formulation of the first aspect of the present disclosure is instilled into an occluded catheter of the patient. As used herein, the terms “treatment” and “treating” include, unless the context dictates otherwise, both the preventive treatment and the therapeutic, i.e. curative, treatment of a subject.

Preferably, the composition is directly instilled into the patient's catheter. Thus, in a third aspect, the present invention relates to a method of treating a patient as herein described. For such purpose, the essentially water-free formulation of the invention may be reconstituted with water. Alternatively, the liquid composition of the present disclosure may be used.

In this method, the tPA is suitable for in-catheter use.

Preferred categories of catheters for practicing said method are catheters, and most preferred are occluded or partially occluded catheters. Administration of the formulation of the present disclosure aims at dissolving occlusions or partial occlusions in catheters. For such purpose, the formulation is instilled directly into the catheter.

Central venous access devices (CVADs) are one category of catheters. CVADs are the most preferred types of catheters in the methods according to the present invention. Briefly, the disclosure also contemplates a method of treating a patient suffering from impaired functionality of a central venous access device (CVAD), comprising administering to the patient the liquid composition of the present disclosure.

Without wishing to be bound to a particular theory, it is envisaged that the tPA acts by targeting fibrin (which causes blood to clot), dissolving the thrombus (blood clot) and restoring function to the central venous access device (CVAD). Thus, preferably, the catheter is a central venous access device (CVAD), and the composition is directly instilled into the CVAD. Preferably, said method is suitable for restoration of functionality of the patient's central venous access device (CVAD).

For the purpose of said method, the composition according to the present invention is preferably provided in a single use vial. To that end, the single use vial preferably comprises the essentially water-free composition according to the second aspect, and is reconstituted prior to administration, whereby the liquid composition according to the first aspect is obtained.

Two main alternative modes of administration are contemplated herein:
In a first embodiment, the administration is a single administration.
In a second embodiment, said administration is a double administration. That is, after a first (single) administration, the administration is repeated one time after an interval of about 60 to 120 minutes, preferably about 90 minutes.

Thus, the tPA is indicated for the restoration of function to CVADs. Preferably, the restoration of function of CVADs as assessed by the ability to withdraw blood. Still preferably the restoration of CVAD functionality is measured as the ability to withdraw 3 mL of blood and/or to infuse 5 mL of saline.

The reconstituted tPA according to present invention is preferably instilled into the catheter. Thereby, the tPA limits systemic exposure because it essentially remains in the catheter, in direct exposure to the clot. Even though a minor fraction may enter the bloodstream, circulating plasma levels are not expected to reach pharmacological concentrations.

The formulation suitable in such method preferably has essentially uncompromised clot lysis activity. Thus, irrespective of whether or not the formulation was freeze-thawed prior to administration to the patient, the patient is administered the active ingredient with essentially uncompromised clot lysis activity. This advantage is achievable specifically with the freeze-dried formulation according to the present disclosure because such formulation is stable in essentially water-free (freeze-dried) form at room temperature.

In the third aspect, in other words, the invention also covers tPA for use in a method of treating a patient as herein described, such as treating a patient suffering from impaired functionality of a catheter, such as a central venous access device (CVAD), wherein the method comprises administering to the patient the liquid composition of the present disclosure. For such purpose, the essentially water-free formulation of the present disclosure may be reconstituted with water.

### Methods of Converting Compositions

In a fifth aspect, the present disclosure also relates to a method of obtaining the composition of the first aspect, wherein the method essentially consists of admixing water to the composition of the second aspect. It is generally preferred that the water is water at a high purity grade, more preferably water for injection (WFI). In particular, sterile water for injection (sWFI) is preferred. Preferably, such method is suitable in the context of the single use vials herein described. This allows shipping of the storage stable composition of the second aspect to the site of use, and conversion of the essentially water-free composition into the liquid composition at said site of use. Without limitation, a typical site of use is an infusion center. Typical infusion centers include clinics and hospitals. The use at said site is normally the administration to a patient, as herein described.

In a sixth aspect, the present disclosure also relates to a method of obtaining the essentially water-free composition of the second aspect, wherein the method essentially consists of removing water from the composition of the first aspect. Specifically, suitable embodiments are described in the examples below, although those should not be construed as limiting.

The compositions are convertible as shown in the following Table 1.

TABLE 1
Schematic overview of the
compositions according to the present invention.

| | Advantage(s) |
|---|---|
| Composition | (non-limiting; for details see text) |
| Formulated Bulk Drug | Freeze-thaw stable |
| Substance (FBDS) | No re-formulation (change of |
| | composition) required; can be directly |
| | aliquoted |
| ↓ aliquot, lyophilize | |
| Essentially water-free composition | Stable at room temperature; |
| (e.g. dry powdered cake) | Reconstitution will yield composition |
| | for in-catheter use |
| ↓ reconstitute (with pre-defined | |
| amount of water) | |
| Ready to use Drug Product | Ready to use |
| (for in-catheter administration) | |

Drug product manufacturing comprises thawing the frozen liquid drug substance, then lyophilize it to produce the essentially water-free composition. The essentially water-free composition is preferably a dry powdered cake in vials (lyophilizate). The liquid formulation prior to lyophilization is defined as the bulk drug substance or BDS for short.

Drug product is produced into a lyophilized form to give it the stable properties in room temperature.

### Kit

In a fourth aspect, the present disclosure also relates to a kit comprising the essentially water free composition of the first aspect as well as water for reconstitution. In particular, the present invention relates to a kit as herein described. To that end, the composition may be commercialized as a kit (drug vial co-packaged with water).

Briefly, such kit may comprise the essentially water-free formulation according to the present invention, as well as an appropriate amount of water for injection (WFI). The formulation and the WFI may be packed together, optionally but preferably together with instructions for use.

However, in some embodiments, it is more preferred that the composition will not be commercialized as a kit (drug vial co-packaged with water). In such case, it is just the drug vial commercially packaged in a box with the Prescribing Information/pamphlet.

The following Examples and Comparative Examples illustrate the invention without limiting its scope.

### EXAMPLES

### Comparative Example 1: Activase (Alteplase Lyophilized Powder)

Alteplase is a recombinant tPA. More specifically, alteplase is the purified tPA glycoprotein of 527 amino acids. Alteplase is obtained using the complementary DNA (cDNA) for natural human tissue-type plasminogen activator (tPA) obtained from a human cell line. The manufacturing process involves secretion of the enzyme alteplase into the culture medium by an established mammalian cell line (Chinese hamster ovary, CHO) into which the cDNA for alteplase has been genetically inserted.

Activase is a lyophilized preparation containing alteplase in arginine phosphate buffer with approximately 0.01% polysorbate 80. It is supplied as a sterile, lyophilized powder. Alteplase, when formulated as Activase lyophilized powder, shows no significant loss of bioactivity or changes in the molecule after storage for more than 4 years at controlled room temperature. Both excipients and pH are considered to contribute to the stability of Activase at room temperature. Degradation of lyophilized Activase however occurred following extended exposure to high temperatures, where the main degradation pathway in the solid state was the formation of higher-molecular-weight species (Nguyen et al., 1993, in Stability and Characterization of Protein and Peptide Drugs: Case Histories, Plenum Press, New York).

In liquid state, alteplase is also prone to cleavage by a protease, purportedly alteplase itself (Nguyen et al., ibid). In reflection of the instability in liquid state, Activase is not commercially available in liquid state, but as a lyophilized powder for reconstitution prior to use.

In summary, Activase lyophilized powder is generally considered stable at room temperature (Nguyen et al., ibid).

### Comparative Example 2: Cathflo (Refrigerated Alteplase Lyophilized Powder)

Cathflo™ Activase® (Genentech, Inc., South San Francisco, CA), first approved by the FDA in 2005, is a lyophilized powder comprising alteplase (see Comparative Example 1), intended for solution for injection and infusion.

According to information provided by the FDA (https://www.accessdata.fda.gov/drugsatfda_docs/label/2018/103172s5260lbl.pdf), 1 vial of Cathflo™ Activase® powder contains 2.2 mg of Alteplase (which includes a 10% overfill), 77 mg of L-arginine, 0.2 mg of polysorbate 80, and phosphoric acid for pH adjustment. Lyophilized Cathflo™ Activase® must be stored at refrigerated temperature (2-8° C./36-46° F.). Cathflo™ Activase® powder is a sterile, white to pale yellow lyophilized powder. (Lindemann et al., ibid).

Cathflo™ Activase® should be reconstituted immediately before use, to a final concentration of 1 mg/ml (Lindemann et al., 2004, Clin. J. Oncol., Nurs., vol. 8, p. 417-419). Each reconstituted vial will deliver 2 mg of Cathflo Activase, at a pH of approximately 7.3.

### Comparative Example 3: Retavase® (Reteplase Lyophilized Powder)

Retavase® is a product marketed by Chiesi USA, Inc. (Cary, NC) and contains reteplase as active ingredient. Reteplase is a deletion mutant of tPA in which certain regions of the wild-type tPA molecule have been deleted (Ross, 1999, Clin. Cardiol., vol. 22, p. 165-171).

### Example 1: Initial Screening of Nine Different Formulations

Based on theoretical evaluation of a known formulation of the active pharmaceutical ingredient reteplase (formulated in Comparative Example 3), suitable conditions (permutations of pH and Tranexamic acid (TA), phosphate and sucrose (together: TAPS) components) were selected and tested with respect to their relevance for stabilizing the active pharmaceutical ingredient (API).

The active ingredient was obtained as reteplase API solution from Wacker, Germany.

In particular, the following was tested:
definition of design of experiment (DoE) test matrices of 9 formulation variants in total
preparation (by dialysis) and analysis of all variants of the test matrices
determination of the colloidal stability (intermolecular interaction, A2) by CG-MALS

The thermodynamic stability of the variants was also evaluated, using nanoDSC for the determination of the onset temperature of denaturation (Tonset).

The following matrix of 9 formulation variants was designed (Table E1). The general behavior of the API regarding pH, tranexamic acid (TA) concentration and sucrose concentration was evaluated. Samples were prepared by dialysis of the API into the corresponding formulation buffer.

The pH was modulated from 6.0 to 7.5. For each pH value two tranexamic acid concentrations (1 mM and 10 mM) and two sucrose concentrations (2% and 8%) were tested. As a ninth variant, the center point with pH 6.75, 5 mM tranexamic acid and 5% sucrose was selected. The buffer (potassium phosphate) concentration was kept constant at 50 mM.

TABLE E1

| | TA | sucrose | | c* |
|---|---|---|---|---|
| Sample | [mM] | (w/w) | pH | [mg/mL] |
| #1 | 1 | 2% | 6.0 | 1.52 |
| #2 | 1 | 2% | 7.5 | 3.33 |
| #3 | 10 | 2% | 6.0 | 1.52 |
| #4 | 10 | 2% | 7.5 | 3.33 |
| #5 | 1 | 8% | 6.0 | 2.98 |
| #6 | 1 | 8% | 7.5 | 4.41 |
| #7 | 10 | 8% | 6.0 | 2.98 |
| #8 | 10 | 8% | 7.5 | 4.41 |
| #9 (CP) | 5 | 5% | 6.75 | 3.36 |

*c is the concentration of the API after dialysis.
In all formulations precipitation was observed - to different extents - which led to a reduced API concentration.
CP = central point for pH.

The colloidal stability of the Reteplase in the formulations variants was evaluated using composition gradient multi angle light scattering (CG-MALS) for the determination of the second virial coefficient A2. Thermodynamic stability of Reteplase in formulation variants was evaluated by nanoDSC for the determination of the onset temperature of denaturation (Tonset).

During dialysis precipitation was observed in all variants. Samples were centrifuged and filtered prior to CG-MALS measurement to remove precipitate, as intermolecular forces can be measured between dissolved molecules only.

Tranexamic acid appeared to have a beneficial effect on the thermodynamic stability of reteplase, however no effect on the colloidal stability could be documented.

Lower sucrose concentrations appear to reduce the onset temperature (Tonset) of the thermal unfolding process. Highest Tonset values beneficial for the thermodynamic stability were determined for variants #4, #7 and #8.

The pH value had only marginal effects on the thermodynamic stability of the API. Variation of pH towards higher values (pH 7.5) showed a clear beneficial effect with regard to colloidal stability.

The nine formulations were evaluated by nano differential scanning calorimetry (Nano DSC), Composition gradient multi-angle light scattering (CG-MALS), CG-MALS measurement, and other analytical methods.

NanoDSC is specifically designed to determine the denaturation temperature and denaturation enthalpy of proteins and other macromolecules in solution with the versatility and precision to perform molecular stability screenings (data not shown) The onset temperature of thermal denaturation, i.e. the starting point of the unfolding transition, is defined as Tonset, the data for which is shown below in Table E3.

An interaction between protein molecules in solution was characterized by changes in their light scattering behavior at different concentrations via CG-MALS. With this series of light scattering measurements the second virial coefficient A2, which is a characteristic parameter to evaluate molecule interactions, was calculated. A2 is a characteristic for a macromolecule and its solvent which describes molecular interactions between the dissolved macromolecules. A negative A2 indicates attractive interactions between molecules of the dissolved substance whereas a positive A2 is characteristic for repulsive interactions between the dissolved protein molecules. The CG-MALS technique is based on a series of differently concentrated macromolecular solutions which are directly injected into the flow cell of a multi-angle light scattering detector. After each injected concentration step the flow is stopped to permit the reaction to reach equilibrium for signal stabilization and subsequent detection. The apparent weight average molecular weight (Mwapp) is determined for each step in the concentration gradient by analyzing the light scattering and concentration data (data not shown).

The results of the thermodynamic stability and colloidal stability evaluation for reteplase are summarized in the following table (Table E3).

TABLE E3
Results of thermodynamic and colloidal stability evaluation

| | TA | sucrose | | c* | A2 [10\hat{ }−4 | Tₒₙₛₑₜ | |
|---|---|---|---|---|---|---|---|
| Sample | [mm] | (w/w) | pH | [mg/mL] | mol * mL/g²] | [° C.] | comment |
| #1 | 1 | 2% | 6.0 | 1.52 | —** | 62 | precipitation |
| #2 | 1 | 2% | 7.5 | 3.33 | −3.5 | 59 | precipitation |
| #3 | 10 | 2% | 6.0 | 1.52 | —** | 63 | precipitation |
| #4 | 10 | 2% | 7.5 | 3.33 | −4.5 | 64 | precipitation |
| #5 | 1 | 8% | 6.0 | 2.98 | —** | 62 | precipitation |
| #6 | 1 | 8% | 7.5 | 4.41 | −2.3 | 60 | precipitation |
| #7 | 10 | 8% | 6.0 | 2.98 | —** | 64 | precipitation |
| #8 | 10 | 8% | 7.5 | 4.41 | −2.2 | 64 | precipitation |
| #9 (CP) | 5 | 5% | 6.75 | 3.36 | −4.4 | 54 | precipitation |

*c is the concentration of the API after dialysis, in formulations #4 to #12 precipitation was observed which led to a reduced API concentration
**A2 could not be calculated, as no stable light scattering values could be measured.

Observed precipitate was of white color and amorphous. Most of the precipitate dissolved upon addition of tranexamic acid. The remaining precipitate (variants #1, #2, #3 and #5) was removed by centrifugation and subsequent filtration using a 0.1 μm syringe filter unit.

The following general observations could be derived from the CG-MALS and nanoDSC measurements (data not shown):

Precipitation of the API was observed during dialysis in all formulation variants. As the concentration of the API decreased significantly after dialysis (Table E3) it is most likely that rPA precipitated. This effect seemed more pronounced at pH 6.0 compared to 7.5, based on the observed API concentration after dialysis, which was higher at pH 7.5 compared to the corresponding variant with pH 6.0.

However, precipitation should not be an issue in the following Examples, since the rPA target concentration is lower at 0.36 mg/mL.

For pH 6.0 variants the CG-MALS data could not be interpreted.

### Conclusions From Example 1 and the Above

All tested variants revealed attractive intermolecular interaction independent of the TA concentration. Only higher sucrose levels seemed to have a slightly beneficial effect.

The thermodynamic stability is enhanced by increasing the tranexamic acid concentration. The onset temperature of the thermal transition rises from about 60° C. at 1 mM TA (e.g. variants #6-#2) to about 64° C. at 10 mM TA (e.g. variants #4-#8).

The pH value had marginal effects on the thermodynamic stability of the API.

Although the thermodynamic stability was slightly higher at a TA concentration of 10 mM, the colloidal stability for both TA concentrations measured are comparable for the 10 variants with 8% sucrose at pH 7.5.

As 60° C. is far from relevant handling conditions, variant #6 can be further tested and considered for development. In other words, the slight advantage of the 10 mM tranexamic acid at 60° C. over the 1 mM tranexamic acid is not expected to immediately affect the stability of the formulations at the handling conditions in a hospital. As a result, a range comprising 1 to 10 mM tranexamic acid is considered suitable.

As sucrose concentrations above 5% lead to a prolonged drying time during lyophilization, the actual sucrose concentration for the stabilization of reteplase (in particular cryo- and lyo-protectant) will be assessed in the subsequent Examples.

Among the initial 9 different formulations of Example 1, the closest formulation to the formulation claimed herein is prototype #2 of Example 1. The main difference is that prototype #2 has 2% sucrose w/w whereas the formulation claimed herein has most preferably 3% sucrose w/w.

### Example 2: Comparison of Different Sucrose Concentrations: Forced Degradation Study by Subjecting the Top 3 Formulation Variants to 3 Conditions of Stress

Based on the data from Example 1 assessing the 9 different formulations, 3 formulations were included in the subsequent Example 2 by subjecting them under forced degradation. The 3 formulations are differentiated by the amount of sucrose (3%, 5%, 8%).

Formulation variants (Table E2-1) were prepared by dialysis against the corresponding formulation buffer. Each formulation buffer was prepared by individually weighing the corresponding components (without PS80-PS80 micelles do not migrate through the dialysis membrane, therefore PS80 was supplemented to the drug substance solution after dialysis) into a glass beaker, dissolution in purified water and subsequent pH adjustment to pH 7.5 using 85 o-phosphoric acid solution.

3 mL of the reteplase API protein bulk solution (c=5.1 mg/mL) were diluted to 15 mL with the corresponding dialysis buffer and transferred into preconditioned (in dialysis buffer) dialysis cassettes. Filled units were incubated in 660 mL of the dialysis buffer at 2-8° C. Three buffer changes were performed (2 hours; 2 hours; overnight). After dialysis the recovered API solution was diluted to the target concentration of 0.36 mg/mL with the corresponding dialysis buffer and supplemented with 10% PS80 stock solution (in dialysis buffer) to 0.9% w/w PS80. The DP solution was sterile filtered and filled into sterile 6R glass vials (2 mL per vial). Vials were stoppered and exposed to the dedicated stress conditions as described below.

Thus, based on the conclusions of Example 1, the variants to be tested in the forced degradation study with 1 mM TA concentration and pH 7.5 assuring for maximum amidolytic activity during administration are the following (Table E2-1):

TABLE E2-1

| | TA | sucrose | |
|---|---|---|---|
| Sample | [mM] | (w/w) | pH |
| #1 | 1 | 3% | 7.5 (26 mM potassium phosphate) |
| #2 | 1 | 5% | 7.5 (26 mM potassium phosphate) |
| #3 | 1 | 8% | 7.5 (26 mM potassium phosphate) |

The objective of Example 2 was: To test the three (3) most promising formulation variants (Table E2-1) with the target concentration of approximately 0.36 mg/mL in a forced degradation study.

The formulation variants of reteplase were to be analyzed prior to and after exposure to selected stress conditions, by analytical means as well as by a representative enzymatic activity assay.

Compounded samples were exposed to the following stress conditions 1)-3) as described below.
1) Forced thermal stress and agitation: Storage at 35° C. for 5 d and 2 weeks at 200 rpm.
2) Exposure to light: For 7.5 h at 750 W/m²/35° C. BST (following ICH Q1B).
3) Freeze-thaw stress: Liquid samples were frozen from room temperature (20° C.) to −50° C. at a controlled freezing rate of 1° C./min and warmed up again to room temperature at a controlled heating rate (1° C./min to simulate bulk freezing conditions). Three cycles were performed successively without holding time.

Prior to and after each time point from each stress condition two liquid samples of each variant were taken for analysis. Vials were visually analyzed for precipitation. Samples were analyzed with respect to turbidity by nephelometric turbidity measurement (following EU Pharmacopoeia, 5.0, 2.2.1), concentration by A280 nm, aggregate status by SE-HPLC, and chemical degradation by RP-HPLC. In addition to analytical evaluation, FDS samples were subject to analysis of enzymatic activity in a designated amidolytic activity assay.

### Conclusion

All formulation variants (Table E2-1) showed a distinct sensitivity of rPA to stress conditions directed to chemical degradation—this was explicitly observed through light exposure and thermal stress (data not shown). No clear correlation between the rPA stability and the sucrose level of the formulation was observed.

These weaknesses would not predict a stable liquid presentation of the drug product (DP). Chemical degradation was not observed for samples exposed to freeze-thaw stress, accounting for suitability for a freeze-dried presentation of the drug product.

All formulation variants showed sufficient retained enzymatic activity after stress exposure. No decrease of the specific amidolytic activity could be determined.

Suitable formulations to move forward into a lyophilized drug product of reteplase would be #1 and #2 whereas the high sucrose level of formulation #3 (8%) might complicate the lyophilization process without providing any added beneficial effect.

Amidolytic activity assay: An existing photometric amidolytic activity assay protocol for Retavase (reteplase API solution) was applied and adapted to this example (rPA). The amidolytic activity assay protocol is based on the release of 4-nitroaniline from the substrate S-2288 (Chromogenix, Cat. No. 82085239) by the amidolytic activity of reteplase API solution under alkaline conditions. The amidolytic activity is calculated in enzyme units [U] from the increase in absorption (ΔA/min) at 405 nm. Over a measuring time of at least 2.5 minutes, the increase in absorption (ΔA/min values) of the reteplase API solution activity is linearly proportional (ranging from 0.05-0.2). Amidolytic activity of FDS samples was tested using this implemented activity test. According to results of method implementation, a concentration between 8 and 22 μg/mL was used to achieve an adequate linear absorption slope over the time without reaching substrate limitation. The amidolytic activity is reflected by the slope. The amidolytic activity remained essentially unaffected upon freeze-thawing (data not shown). All samples subjected to increased temperature (35° C.) showed a significant increase in the amidolytic activity, which is potentially an effect of the splitting of the kringle domain from rPA, to give a higher molarity of proteolytic active species: kringle- and protease-domains (data not shown).

RP-HPLC: Reverse phase HPLC analysis is used to determine the two-chained protein content in the reteplase API solution. The reteplase API is being denatured and reduced. This reduction opens the disulphide bridges in the molecule. During this process, the two-chained repletase molecules are split off into the Kringle and protease domains and during the HPLC the domains are separated into the Kringle peaks and the protease peaks (main peaks). Intact reteplase molecules do not split off any Kringle domains and those molecules run only as main peaks (protease peaks) in the HPLC. All variants showed sensitivity of the reteplase molecule to chemical degradation when subject to light exposure and thermal stress. These weaknesses would not predict a stable liquid presentation of the DP. However, there were no major differences between the three formulations were observed after freeze-thawing.

### Example 3: Thermal Properties and Feasibility of Lyophilization

Based on the results of Example 2, the following 2 preferred formulation variants of rPA were further evaluated for a lyophilized drug product presentation (2.0 mL fill in 6R vial):

#1: 0.35 mg/mL rPA, 26 mM potassium phosphate, 1 mM tranexamic acid, 3% (w/w) sucrose) pH 7.5
#2: 0.35 mg/mL rPA, 26 mM potassium phosphate, 1 mM tranexamic acid, 5% (w/w) sucrose) pH 7.5

This example is aimed to characterize the thermal properties of the lyophilization solutions by differential scanning calorimetry (DSC) and to subsequently, based on the results of the DSC measurements. The evaluation was conducted at different sucrose percentages to monitor the collapse behavior of each formulation variant during lyophilization in a multistep critical pressure scan (CPS), to monitor the collapse behavior at different pressure levels between 50 μbar and 590 μbar at a shelf temperature of 0° C.

The formulation candidates #1 and #2 were prepared in the respective concentration of 0.35 mg/mL rPA. The DSC measurements revealed two thermal events. For both formulations #1 and #2, the onset of the second order thermal event or glass transition point Tg, was detected around −33.7° C. (corresponding vapor pressure: 0.27 mbar, data not shown). The critical pressure scan showed a collapse of the lyo cake between 50 μbar and 100 μbar in both formulation variants #1 and #2. Based on these findings a chamber pressure of 50 μbar was selected for the first feasibility lyophilization run.

Further, lyophilization feasibility of the samples was to be evaluated and samples were to be freeze dried using a conservative prototype lyo-cycle. To that end, a first feasibility lyophilization run was performed that aimed to show in principle that the designated formulation variant itself is suitable to be freeze dried with sufficiently safe conditions. The formulation candidates #1 and #2 were prepared in the respective concentration of 0.35 mg/mL rPA. One conservative prototype lyo-cycle was performed considering the above findings. The prototype lyophilization cycle was performed successfully.

The lyophilized drug product was analyzed with the following methods:
visual appearance, documented by macro photography
reconstitution behavior (in 2 mL purified water)
residual water content using a generic Karl-Fischer oven method
chemical analysis of reconstituted vials: concentration by UV280 nm, turbidity by nephelometric turbidity measurement, aggregate status by SE-HPLC, chemical degradation by RP-HPLC.

For both formulation variants, acceptable lyo-cakes were obtained proving the suitability of the selected formulation variants for lyophilization. The sample analysis showed no discernible sign of rPA degradation during lyophilization.

In conclusion, both formulation variants have proven suitable for lyophilization using a conservative lyophilization cycle. Lyophilizates showed intact lyo-cakes which reconstitute spontaneously and completely in <10 seconds. Turbidity values were in an acceptable range of ≤5 NTU. Residual water content was well within the generally acceptable humidity level in lyophilized biopharmaceuticals of 1-5% (in accordance with the guidelines). Neither any degradation nor any tendency for aggregation of the API in the reconstituted samples could be observed. Compared to variant #2 (with 5% sucrose) also 3% sucrose (variant #1) proved sufficient to generate an intact lyo-cake without any deficiencies. With variant #1 being able to accomplish the same final product characteristics with less material, formulation variant #1 with 3% sucrose (w/w) was deemed the most appropriate candidate to move forward in development (e.g. to WPS, lyo cycle development).

### Example 4: Lyophilization of the Selected Formulation

As described in the preceding examples, the 3 formulations (Example 2) were then narrowed down to 2 formulations (3% versus 5% sucrose) and subjected through a thermal characterization study wherein formulation with the 3% sucrose was ultimately chosen as the formulation to use for the subsequent study of lyophilization cycle development: the objective of this development work was to develop an efficient and robust lyophilization process for the selected final TAPS formulation variant #1 within three development lyophilization cycles:
#1: 0.35 mg/mL rPA, 26 mM potassium phosphate, 1 mM tranexamic acid, 3% (w/w) sucrose, 0.09% PS80 (polysorbate 80) (w/w), pH 7.5

The sample was prepared in accordance with the preceding examples.

A robust and efficient lyophilization cycle (optimized process parameters for freezing, sublimation and secondary drying) for the best formulation selected from Example 3 was to be developed within three development lyophilization cycles based on the results of Example 3.

The goals of the lyophilization cycle development include the following: to develop a cycle that gives (a) a stable cake and (b) is stable in analytics.

The API rPA was to be dialyzed into the selected formulation system (#1, as above) in the appropriate amount (0.70 mg amount per vial; 0.35 mg/mL; 2.0 mL fill volume per vial). For each cycle a mixed batch was to be used: about 30 samples ‘active’ and 50 test samples ‘placebo’ were to be filled per run and processed. Formulated bulk solutions (active and placebo) were to be compounded. Bulk solutions were to be filled into cleaned and heat-sterilized 6R glass vials and partially stoppered with autoclaved and dried lyo stoppers and loaded into a freeze dryer and lyophilized. Placebo vials were to be surrounding the active samples. Placebo vials (filled with final formulation buffer only) were placed around active vials to simulate large scale conditions (slower drying anticipated for interior/thermally-shielded vials vs. those not thermally shielded on the tray periphery).

In-vial temperatures were to be monitored by in-vial micro-thermocouples. Chamber pressure was to be monitored by capacitance pressure sensor and Pirani pressure sensor and recorded via on-line data acquisition. Process analytical data were to be collected and evaluated. The lyophilization parameters were to be systematically adapted and optimized.

The lyophilized vials were to be closed after venting with nitrogen. Pressure regulation was managed via vacuum and dosing valve (nitrogen injection). After completion of secondary drying, or primary drying in single step cycle respectively, vials were closed at a pressure of 800 mbar under nitrogen atmosphere.

Active samples were to be analyzed with the following methods:
visual appearance, documented by macro photography
reconstitution behavior
residual water content using a generic Karl-Fischer oven method

Remainder spare vials were to be stored.

Based on Example 3 and on further preliminary experiments (not shown), a suitable lyophilization cycle was developed as follows: The process parameters applied for the single step lyophilization cycle are listed in Table E4-1.

TABLE E4-1
Process parameters of the lyophilization cycle

| | | | Ice | | | Cumu- |
|---|---|---|---|---|---|---|
| | | Shelf | condenser | Pressure | Time | lative |
| Step | | temperature | temperature | [Pirani] | step | time |
| # | Description | [° C.] | [° C.] | [mbar] | [h:min] | [h:min] |
| 1 | Loading | 25 | — | atm | 00:10 | 0:10 |
| 2 | Freezing | −50 | — | atm | 01:15 | 1:25 |
| | ramp | | | | | |
| 3 | Freezing | −50 | — | atm | 04:00 | 5:25 |
| 4 | Vacuum | −50 | −80 | 0.05 | 00:30 | 5:55 |
| | Adjustment | | | | | |
| 5 | Primary | 30 | −80 | 0.05 | 07:00 | 12:55 |
| | Drying | | | | | |
| | ramp | | | | | |
| 6 | Primary | 30 | −80 | 0.05 | 40:00 | 52:55 |
| | Drying | | | | | |
| 7 | Venting & | 5 | — | 750 | 00:05 | 53:00 |
| | closing | | | | | |

In this lyophilization cycle the inflection point is approximately at 18.5 h total process time/5.5 h primary drying time. The results of the thermocouples measurements show almost no significant variance between the positioning of the vials in respect to sublimation time (thermocouples 1 and 2 of center (1) and edge (2) vials) (Data not shown). All sample vials were intact without glass breakage.

The obtained Lyophilisate is dissolved within 10-12 seconds with water for injection (WFI).

Overall, this lyophilization cycle was determined to be particular suitable for obtaining the essentially water-free composition according to the present invention: in particular, the developed cycle gives (a) a stable cake and (b) is stable in analytics. The cycle selected in this example fulfils these requirements.

### Example 5: The Freeze-Dried Formulation is Stable Even at Elevated Temperatures and Reconstitution

Lyophilizates were stored for several weeks (0 weeks, 0 w; 4 weeks, 4 w, 8 weeks, 8 w; 12 weeks, 12 w) at different temperatures and analyzed.

The visual appearance of lyophilized vials was recorded by macro-photography. All lyophilizates showed intact lyo-cakes without major defects.

Concentration: The rPA concentration was calculated according to Lambert-Beer's law using an extinction coefficient of 1.69 mL/(mg*cm). Two vials per timepoint were analyzed in duplicate. As a result, it was determined that concentrations remained throughout the storage period in an acceptable range according to the target value of 0.35 mg.

Residual water content (Karl Fischer titration): After lyophilization samples for residual water content determination were prepared by transferring one lyo cake per sample into a Karl Fischer vial, which was sealed immediately. The water content of the lyo cakes was determined by heating the sample to 90° C. in a Karl Fischer oven and transferring evaporated water into the coulometer with a flush of dry nitrogen. Two vials were analyzed in a single measurement each (one sample preparation each). For blank correction, empty vials were used. Results of residual water content are compiled in Table E5-1. Both T0 samples' residual water values are well below the generally accepted residual water content of lyophilizates of 2%. A residual moisture content below 2% for a lyophilized drug product is generally considered as safe (Daukas, L. A., Trappler, E. H., 1998. Assessing the quality of lyophilized parenterals. Pharm. Cosmetic Quality 2, 21-25). A slight but negligible higher moisture content at T8 and T12 weeks was evident. This likely resulted from the equilibrium moisture of the stoppers (assumption based on the correlation of increase with storage temperature).

TABLE E5-1
Residual water content of lyophilizates

| | Residual water content [%] | | | | | | | | | |
|---|---|---|---|---|---|---|---|---|---|---|
| Sample | | T4 weeks | | | T8 weeks | | | T12 weeks | | |
| vial | T0 | 2-8° C. | 25° C. | 40° C. | 2-8° C. | 25° C. | 40° C. | 2-8° C. | 25° C. | 40° C. |
| vial 1 | 0.73 | 0.90 | 0.95 | 1.20 | 1.12 | 1.27 | 1.32 | 0.97 | 1.18 | 1.22 |
| vial 2 | 0.75 | 0.86 | 1.02 | 1.05 | 0.98 | 1.34 | 1.34 | 1.14 | 1.10 | 1.22 |

Two lyophilizates of T0, T4 w, T8 w and T12 w were reconstituted with 2.0 mL purified water. The reconstitution time was monitored. All lyophilizates at timepoints T0, T4 w, T8 w and T12 w showed spontaneous and complete dissolution of the lyo cakes within 10 seconds.

Nephrolometric turbidity: Turbidity was measured in the undiluted samples using a calibrated nephelometer after reconstitution. As a result, it was determined that turbidity values were very low for all samples. No visual turbidity could be observed after reconstitution of lyophilized vials at sample timepoints T0 to T12 weeks.

Analytical evaluation by RP-HPLC of lyophilizates after reconstitution confirmed stability of the main peak over the storage period (data not shown).

The aggregate status of each timepoint and storage condition was determined by SE-HPLC, and was found to be essentially stable.

Amidolytic activity was tested using the implemented activity test as described in Example 2. The amidolytic activity is reflected by the slope. Results of activity assay are summarized in Table E5-2.

TABLE E5-2
Results of the specific activity assay of samples. Values are mean values of nine
measurements per sample. The samples were prepared by dilution with Tris buffer
to an rPA concentration of 8.75 μg/mL.

| | mean [KU/mg]; n = 9 | | | | | | | | | |
|---|---|---|---|---|---|---|---|---|---|---|
| | | T4 weeks | | | T8 weeks | | | T12 weeks | | |
| Sample | T0 | 2-8° C. | 25° C. | 40° C. | 2-8° C. | 25° C. | 40° C. | 2-8° C. | 25° C. | 40° C. |
| pre- | 459 | — | — | — | — | — | — | — | — | — |
| lyophilization | | | | | | | | | | |
| sample | | | | | | | | | | |
| vial 1 | 436 | 459 | 481 | 454 | 469 | 465 | 478 | 484 | 479 | 477 |
| vial 2 | 481 | 482 | 477 | 501 | 470 | 473 | 489 | 481 | 477 | 475 |

Amidolytic activity was consistent over the course of the study for all samples.

### Example 6: Overview Comparison With Commercial Retavase

One goal behind the formulation of the present disclosure is a commercially suitable formulation that it is similar to diluted Retavase in terms of constituents, but superior in stability and other parameters. To that end, the inventor has developed a stable formulation that has the same excipients.

For an easier comparison, the Table E6-1 below summarizes the formulation of commercial 081 versus 081 clinical (diluted Retavase) versus Retavase (as utilized for its approved indication on the market) and Table E6-2 reports further formulations according to the invention:

TABLE E6-1
Comparison of various TAPS-containing formulations (in 2 mL)

| | Internal Identifier: | | |
|---|---|---|---|
| | A | B | |
| | Present Invention, | For clinical trial | C |
| | Example | (Example 7) (diluted | Retavase |
| Formulation | Embodiment | Retavase) | Commercial |
| Reteplase | 0.7 mg | 0.7 mg | 17.4-18.1 mg |
| | | | (5460 |
| | | | mg weight) |
| Tranexamic acid | 0.31 mg | 0.32 mg | 8.00 mg |
| Sucrose | 61 mg | 14.0 mg | 350 mg |
| Polysorbate 80 | 1.9 mg | 0.20 mg | 5.0 mg |
| Dipotassium | 8.36 mg | 5.24 mg | 131 mg |
| phosphate | | | |
| Phosphoric acid | 0.69 mg | 1.97 mg | 49.3 mg |
| 85% | | | |

Formulation A corresponds to 0.09% PS80 (w/w)

Table E6-2: Other examples of TAPS-containing formulations

Formulation A2-0.35 mg/mL Reteplase, 26 mM potassium phosphate, 1 mM tranexamic acid, 3% (w/w) sucrose, 0.0675% PS80 (w/w), pH 7.5 (Repletase:PS 80 ratio 0.52:1)
Formulation A3-0.35 mg/mL Reteplase, 26 mM potassium phosphate, 1 mM tranexamic acid, 3% (w/w) sucrose, 0.045% PS80 (w/w), pH 7.5 (Repletase:PS 80 ratio 0.78:1)
Formulation A4-0.35 mg/mL Reteplase, 26 mM potassium phosphate, 1 mM tranexamic acid, 3% (w/w) sucrose, 0.02% PS80 (w/w), pH 7.5 (Repletase:PS 80 ratio 1.75:1)
Formulation A5-0.35 mg/mL Reteplase, 26 mM potassium phosphate, 1 mM tranexamic acid, 3% (w/w) sucrose, 0.01% PS80 (w/w), pH 7.5 (Repletase:PS 80 ratio 3.5:1)

As described above, the formulations A, A1-A4 of the present disclosure were developed as the most stable formulations, starting from initially 9 different prototypes. In particular the relative amounts of sucrose and/or of polysorbate 80 drastically distinguish the formulation of the present disclosure from diluted and undiluted preparations of Retavase as previously known.

### Example 7: Clinical Trial in Subjects With Dysfunctional Non-Hemodialysis Central Venous Access Devices (CVADs)

In a phase III, multinational, multicenter, randomized, double-blind, parallel-group, active and placebo-controlled study to examine the administration of a TAPS-buffered reteplase formulation comprising 0.7 milligrams (mg) (0.4 units) per 2 milliliter (mL) of reteplase (diluted Retavase), versus placebo or alteplase in subjects with dysfunctional non-hemodialysis Central Venous Access Devices (CVADs).

Participants will receive 1 or 2 doses of Formulation B (see Example 6), 0.7 milligrams (mg) (0.4 units) per 2 milliliter (mL) directly into the catheter lumen. Participants will receive the first dose at minute (min) 0, and the second dose, if needed, at min 90.

During the study, the treatment period will consist of one visit which may take place on the same day as screening or on the following day. After meeting all inclusion criteria, subjects will be randomized in a 9:1:6 ratio of formulation A, A1-A5:placebo:alteplase. There will be a follow-up assessment performed on Day 30 (±2 days) after treatment with study drug.

Routine blood pressure measurement, heart rate and urine pregnancy test will be performed before enrollment in the study. Safety, including Treatment Emergent AEs (TEAEs), Adverse Drug Reactions (ADRs) and Adverse Events of Special Interest (AESI), will be recorded throughout the study. The end of the trial is defined as the last follow-up contact of the last subject to receive study drug in the trial.

The clinical trial is generally indicative of the effects of a TAPS-buffered reteplase formulation comprising 0.7 milligrams (mg) (0.4 units) per 2 milliliter (mL).

The formulation of the present invention (see e.g. Formulations A, A1-A4 Example 6) is superior to the diluted reteplase of (Formulation B, Example 6) at least in the various stability parameters discussed in the present disclosure, including in terms of storage stability at room temperature and in terms of preparation.

### Example 8: Instructions for Use in a Clinical Setting

Instructions for use in a commercial setting may for example include the following, or similar, instructions. The following is provided as illustrative example.
1. Reconstitute the essentially water-free composition according to the present invention with water for injection (WFI), and inspect the product prior to administration for foreign matter and discoloration.
2. Withdraw 2 mL (2 mg) of solution from the reconstituted vial.
3. Instill the appropriate dose of tPA formulation into the occluded catheter.
4. After 30 minutes of dwell time, assess catheter function by attempting to aspirate blood. If the catheter is functional, go to Step 7. If the catheter is not functional, go to Step 5.
5. After 90 minutes of dwell time, assess catheter function by attempting to aspirate blood and catheter contents. If the catheter is functional, go to Step 7. If the catheter is not functional, go to Step 6.
6. If catheter function is not restored after one dose of the formulation of the present invention, a second dose of equal amount may be instilled. Repeat the procedure beginning with Step 1 under Preparation of Solution.
7. If catheter function has been restored, aspirate 4-5 mL of blood in patients ≥10 kg or 3 mL in patients <10 kg to remove the tPA formulation.

### Example 9: Development of Two-Step Lyophilization Cycle

As described in Example 4, a single step lyophilization cycle capable of yielding a drug product which is (a) a stable cake and (b) is stable in analytics was developed. Following the development of the more single step lyophilization cycle, a subsequent study was executed to also develop a more conventional two-step lyophilization cycle. As described in the preceding examples, the 3% sucrose formulation was ultimately chosen as the formulation to use for the subsequent study of lyophilization cycle development. The objective of this development work was to develop an efficient and robust two-step lyophilization process for the selected final TAPS formulation variant #1 (#1: 0.35 mg/mL rPA, 26 mM potassium phosphate, 1 mM tranexamic acid, 3% (w/w) sucrose, 0.09% PS80 (polysorbate 80) (w/w), pH 7.5) within four development lyophilization cycles.

The goals of the lyophilization cycle development include the following: to develop a cycle that gives (a) a stable cake and (b) is stable in analytics.

Formulated bulk solutions (active and placebo) were compounded. The API rPA was adjusted to the target formulation and target concentration (0.70 mg amount per vial; 0.35 mg/mL; 2.0 mL fill volume per vial) with the appropriate amount of adjustment buffer. Bulk solutions were sterile filtered and filled into cleaned and heat-sterilized 6R vials and partially stoppered with autoclaved and dried lyo stoppers, followed by freeze-drying and lyophilization.

For each cycle a mixed batch was used: about 10 samples ‘active’ and 350 test samples ‘placebo’ were filled per run and processed. Placebo vials (filled with final formulation buffer only) were placed around active vials to simulate large scale conditions (slower drying anticipated for interior/thermally shielded vials vs. those not thermally shielded on the tray periphery).

In-vial temperatures were monitored by in-vials micro-thermocouples. Pressure regulation was managed via vacuum and dosing valve (nitrogen injection). Chamber pressure was monitored by capacitance pressure sensor and Pirani pressure sensor and recorded via on-line data acquisition. The end of sublimation was detected by capacitive pressure sensor/Pirani pressure sensor difference. Process analytical data was collected and evaluated. The lyophilization parameters were systematically adapted and optimized.

The lyophilized vials were closed after venting with nitrogen. After completion of secondary drying, vials were closed at a pressure of 800 mbar under nitrogen atmosphere.

Active samples were analyzed with the following methods:
visual appearance, documented by macro photography
reconstitution behavior
residual water content using a generic Karl-Fischer oven method

Based on Examples 3 and 4, and on further preliminary experiments (not shown), a suitable lyophilization cycle was developed as follows: the process parameters applied for the two-step lyophilization cycle are listed in Table E9.

TABLE E9
Process parameters applied for the two-step lyophilization cycle

| | | | Ice | | | Cumu- |
|---|---|---|---|---|---|---|
| | | Shelf | condenser | Pressure | Time | lative |
| Step | | temperature | temperature | [Pirani] | Step | Time |
| # | Description | [° C.] | [° C] | [mbar] | [h:min] | [h:min] |
| 1 | Loading | 25 | — | atm | 00:10 | 0:10 |
| 2 | Freezing, | −50 | — | atm | 01:50 | 2:00 |
| | ramp | | | | | |
| 3 | Freezing | −50 | — | atm | 04:00 | 6:00 |
| 4 | Vacuum | −50 | <−65 | 0.04 | 00:30 | 6:30 |
| | adjustment | | | | | |
| 5 | Primary | −25 | <−65 | 0.04 | 00:40 | 7:10 |
| | drying, ramp | | | | | |
| 6 | Primary | −25 | <−65 | 0.04 | 62:00 | 69:10 |
| | drying | | | | | |
| 7 | Secondary | 30 | <−65 | 0.04 | 01:20 | 70:30 |
| | drying, ramp | | | | | |
| 8 | Secondary | 30 | <−65 | 0.04 | 16:00 | 86:30 |
| | drying | | | | | |
| 9 | Venting and | 5 | — | 750 | 00:05 | 86:35 |
| | closing | | | | | |

In this lyophilization cycle, the inflection point is approximately at 44 h total process time/37 h of primary drying time. The lyophilization was controlled by capacitance pressure sensor (MKS). All sample vials were intact without glass breakage.

The conservative two-step lyophilization cycle for formulation A, A1-A5 DP resulted in defect free lyophilizates with uniform color and texture. The obtained lyophilizates were dissolved within 10 seconds, using purified water. The achieved residual water content of lyophilizate samples were below 1% which is well below the acceptable limit of 5% for a pharmaceutical lyophilized drug product.

Overall, this lyophilization cycle was determined to be particularly suitable for obtaining the essentially water-free composition according to the present invention: in particular, the developed cycle gives (a) a stable cake and (b) is stable in analytics. The cycle selected in this example fulfils these requirements.

1. A liquid composition comprising:
i. about 0.15 to 0.25 units (U)/mL tissue plasminogen activator (tPa),
ii. about 1 mM to 10 mM tranexamic acid,
iii. 2.5 to 5.0% (w/w) sucrose; (preferably about 3.0% (w/w),
iv. optionally a nonionic surfactant,
v. optionally a pH buffering agent;
vi. wherein the formulation has a pH of 6.0 to 8.0.

2. The composition of claim 1, wherein the tissue plasminogen activator (tPA) is reteplase.

3. The composition of claim 2, wherein reteplase is contained at an amount of 0.2 units (U)/mL.

4. The liquid composition of any one of previous claim, wherein tPa is present at about 0.3 to 0.4 mg/mL.

5. The liquid composition of any one of previous claim, wherein tranexamic acid is present at about 1 mM.

6. The liquid composition of any one of previous claim, comprising a nonionic surfactant, wherein the nonionic surfactant is polysorbate 80 (PS80).

7. The liquid composition of claim 6 wherein PS80 is comprised at an amount of between 0.001 (w/w) and 0.1% (w/w).

8. The liquid composition of claim 6 or 7 wherein PS80 is comprised at an amount selected from the group consisting of: 0.01% (w/w), 0.02% (w/w), 0.03% (w/w), 0.04% (w/w), 0.045% (w/w), 0.06% (w/w), 0.0675% (w/w), 0.07% (w/w), 0.08% (w/w) and 0.09% (w/w).

9. The liquid composition of any one of previous claim, comprising a pH buffering agent, which is a potassium phosphate buffering system, and preferably comprises 20-30 mM potassium phosphate, more preferably about 26 mM potassium phosphate.

10. The liquid composition of any one of previous claim having a pH of 7.0 to 8.0, preferably 7.5.

11. The liquid composition of any one of previous claim, wherein the composition is comprised in a single use vial.

12. The liquid composition any one of previous claim, wherein the composition is stable at room temperature for at least 3 months, preferably at least 6 months.

13. An essentially water-free composition obtainable by removing water from the composition any one of previous claim, preferably by lyophilization.

14. The composition of claim 13, wherein the composition is in the form of a powder.

15. A single use vial comprising the composition of claim 13.

16. The single use vial of claim 15, wherein the single use vial has a volume capacity of 2 to 15 mL.

17. The single use vial of claim 15, wherein the single use vial comprises approximately 2 ml of said composition.

18. A method of treating a patient suffering from impaired functionality of a catheter, wherein the method is characterized in that the liquid formulation of any one of claims 1 to 12 is instilled into an occluded catheter of the patient.

19. The method of claim 18, wherein the catheter is a central venous access device (CVAD), and wherein the composition is directly instilled into the CVAD.

20. The method of claim 19, wherein the method is suitable for restoration of functionality of the patient's central venous access device (CVAD).

21. The method of claim 18, wherein the liquid formulation is provided in a single use vial.

22. The method of claim 18, wherein said instillation is a single administration.

23. The method of claim 18, wherein said instillation is repeated one time after an interval of about 60 to 120 minutes, preferably about 90 minutes.

24. A kit comprising the unit dosage composition of any one of claim 13 or 14 and water for reconstitution.

25. A method of reconstituting the composition of any one of claims 1 to 12, wherein the method essentially consists of admixing water to the composition of claim 13.

26. Use of the composition of any one of claims 1 to 12 or the composition of claim 13 to restore the functionality of a catheter.
